(19) United States
(12) Patent Application Publication
(10) Pub. No.: US 2008/0031825 A1
(43) Pub. Date: Feb. 7, 2008

(54) Pharmaceutical Compositions Comprising Effervescent Agents And Fenofibrate

(76) Inventors: Yisheng Chen, Gurnee, IL (US); Yihong Qiu, Vernon Hills, IL (US); Thomas L. Reiland, Surprise, AZ (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/573,932

(22) PCT Filed: Aug. 5, 2005
(86) PCT No.: PCT/US05/27901

§ 371 Date: Aug. 21, 2007

Related U.S. Application Data
(60) Provisional application No. 60/603,198, filed on Aug. 20, 2004.

(51) Int. Cl. A61K 31/216; A61K 9/20; A61P 3/06

(52) U.S. Cl. 424/43; 424/451; 424/464; 514/533

(57) ABSTRACT

A pharmaceutical composition comprising at least one effervescent agent and a fibrate and a process for making such composition. The fibrate is fenofibrate and the effervescent agent are in a dosage form. The dosage form increases dissolution and absorption of fenofibrate in biological conditions where the form contacts acidic gastric fluid after oral administration.

### FIELD OF THE INVENTION

The present invention relates to novel compositions comprising lipid-regulating agents.

### BACKGROUND OF THE INVENTION

2-[4-(4-chlorobenzoyl)phenoxy]-2-methyl-propanoic acid, 1-methylethyl ester, also known as fenofibrate, is representative of a broad class of compounds having pharmaceutical utility as lipid-regulating agents. More specifically, this compound is part of a lipid-regulating agent class of compounds commonly known as fibrates, and is disclosed in U.S. Pat. No. 4,058,552.

Fenofibrate has been prepared in several different formulations, c.f., U.S. Pat. No. 4,800,079 and U.S. Pat. No. 4,895,726. U.S. Pat. No. 4,895,726 discloses a co-micronized formulation of fenofibrate and a solid surfactant.

U.S. Pat. No.4,961,890 discloses a process for preparing a controlled release formulation containing fenofibrate in an intermediate layer in the form of crystalline microparticles included within pores of an inert matrix. The formulation is prepared by a process involving the sequential steps of dampening an inert core with a solution based on a binder, then projecting fenofibrate microparticles in a single layer onto a dampened core, and thereafter drying and repeating the three steps in sequence until the intermediate layer is formed.

European Patent Application No. EP0793958A2 discloses a process for producing a fenofibrate solid dosage form utilizing fenofibrate, a surface active agent and polyvinyl pyrrolidone in which the fenofibrate particles are mixed with a polyvinyl pyrrolidone solution. The thus obtained mixture is granulated with an aqueous solution of one or more surface active agents, and the granulate thus produced is dried.

PCT Publication No. WO82/01649 discloses a fenofibrate formulation having granules that are comprised of a neutral core that is a mixture of saccharose and starch. The neutral core is covered with a first layer of fenofibrate, admixed with an excipient and with a second microporous outer layer of an edible polymer.

U.S. Pat. No. 5,645,856 discloses the use of a carrier for hydrophobic drugs, including fenofibrate, and pharmaceutical compositions based thereon. The carrier comprises a digestible oil and a pharmaceutically-acceptable surfactant component for dispersing the oil in vivo upon administration of the carrier, which comprises a hydrophilic surfactant, said surfactant component being such as not to substantially inhibit the in vivo lipolysis of the digestible oil.

The prior art processes obtain small particles of fenofibrate by the use of co-micronization steps of the drug with a surfactant. These resulting formulations may not have the maximized dissolution rate.

It is an object of the present invention to provide rapid dissolution of lipid-regulating agents, more preferably fenofibrate, having enhanced dissolution and absorption characteristics than those particles of such agents prepared by prior art techniques.

### BRIEF SUMMARY OF THE INVENTION

The present invention is directed to a pharmaceutical composition and a process for preparing a composition of a lipid-regulating agent with enhanced dissolution and absorption characteristics.

The lipid-regulating agent is a fibrate. Preferably, the fibrate is fenofibrate and is mixed with an effervescent agent or other gas generating material in a solid dispersion. This results in a composition having enhanced dissolution and bioavailability characteristics, when compared to a composition prepared by prior art techniques.

More particularly, the present process comprises the steps of mixing an effervescent agent, sodium bicarbonate for example, and a disintigrant (optionally with other excipients) with molten fenofibrate, followed by congealing to form a solid particle dispersion of sodium bicarbonate and disintigrant in fenofibrate. The solid dispersion is then milled to form powders that are suitable for encapsulation or tabletting.

The gas generating material can be a carbonate such as sodium carbonate, sodium bicarbonate, potassium carbonate, potassium bicarbonate, sodium sesquicarbonate, sodium glycine carbonate, L-lysine carbonate, arginine carbonate, calcium carbonate, or other materials that are safe for human use.

Other expedients can be disintigrants, fillers, lubricants, antiadherents, or other expedients that are well known in the art.

The solid dispersion can be prepared by a melt-congealing process that includes a rotary atomization process, an extrusion process, or melt granulation process, a coating process, or other processes that are well known in the art.

The finished oral dosage form may be prepared by techniques well-known to those skilled in the art by milling the mixture, mixing the resultant particles with excipients, and filling or compressing to form the finished oral dosage form, preferably as a tablet, capsule, or a suspension. Because the present invention does not contain acid, the formulation will not release carbon dioxide until the dosage form reaches the acidic gastric fluid, which differentiates the current invention from conventional effervescent formulations containing acids.

The formulation thus produced may be administered directly, diluted into an appropriate vehicle for administration, encapsulated into hard gelatin shells or capsules for administration, compressed into tablets for administration, or administered by other means obvious to those skilled in the art.

The objective of the present invention is to increase the dissolution or absorption of fenofibrate in a biological condition such as in the gastric fluid of a patient. The fundamental principle of the invention is to form an effervescent system of fenofibrate so that the dissolution of the drug will be increased upon contacting the acidic gastric fluid after oral administration. The increased dissolution will then lead to the increased drug absorption for the lowering of lipids.

### BRIEF DESCRIPTION OF THE DRAWING

FIG. 1 is a graph showing the dissolution characteristics of a reference composition compared to a composition of the present invention.

### DETAILED DESCRIPTION OF THE INVENTION

The bulk lipid-regulating agent can be prepared by any available method, as for example the compound fenofibrate may be prepared by the procedure disclosed in U.S. Pat. No. 4,658,552, or the procedure disclosed in U.S. Pat. No. 4,739,101, both incorporated by reference herein.

There are different types of materials that can be used to produce the effervescent effects according to the present invention. The most commonly used materials are carbonates, which may include sodium carbonate, sodium bicarbonate, potassium carbonate, potassium bicarbonate, sodium sesquicarbonate, sodium glycine carbonate, L-lysine carbonate, arginine carbonate and calcium carbonate, or other gas generating materials that are suitable for human use.

Additionally, other effervescent materials comprising a gas on an absorbent may be used. An example is carbon dioxide absorbed on zeolite aluminosilicate.

Other excipients such as disintigrants, fillers, lubricants, antiadherents, or other excipients well known in the art can be used.

Solid dispersions can be prepared by a melt-congealing process including a rotary atomization process, an extrusion process, a melt granulation process, a coating process, a milling process or other processes well known in the art.

The finished oral dosage form may be prepared by techniques well-known to those skilled in the art by sizing the mixture, dry blending the resultant particles with excipients, and filling or compressing to form the finished oral dosage form, preferably as a tablet, a capsule, or a suspension.

A pharmaceutical composition comprising fenofibrate without an effervescent agent is prepared by a manufacturing process well known in the art as shown in Example 1. A pharmaceutical composition of the present invention comprising fenofibrate and an effervescent agent is prepared as shown in Example 2. Both examples are compared in particle size analysis and in vitro dissolution tests.

The invention will be understood more clearly from the following non-limiting representative examples:

### EXAMPLE 1

Fenofibrate (64.0 g) is melted in a beaker on a hot plate. Sodium croscamellose (16.0 g) is added and mixed. The mixture is then poured onto a glass tray and allowed to cool to ambient temperature. The resulting solid is milled using a Fitzmill with a 0.065 inch screen opening. The milled material is then weighed. For each ten (10) g of the milled material, 5.96 g of lactose monohydrate and 0.04 g of silicon dioxide is added. The materials are mixed well. The resulting mixture is allowed to pass through a 50-mesh screen. From the resulting blended powder, 400 mg of the powder is filled into size 00 hard gelatin capsules, which will provide 200 mg of fenofibrate per capsule.

### EXAMPLE 2

Fenofibrate (12.0 g) is melted in a beaker on a hot plate. Both sodium croscamellose (4.0 g) and sodium bicarbonate (4.0 g) are added and mixed. The mixture is then poured onto a glass tray and allowed to cool to ambient temperature. The resulting solid is milled using a Fitzmill with a 0.065 inch screen opening. The milled material is then weighed. From the resulted milled powder, 333 mg of the powder is filled into size 00 hard gelatin capsules, which will provide 200 mg of fenofibrate per capsule.

Analysis and dissolution tests were performed using the products of Example 1 and Example 2 above. Particle size distribution of the milled granules, not including the lactose and silicon dioxide, were measured using a Sympatec Helos system.

The in vitro dissolution rate of the capsules was tested using USP apparatus II. The test conditions were: paddle speed at 50 rpm, dissolution medium of 50 mM SDS in 0.1N HCl solution and temperature at 37 degrees Celsius. Dissolution samples were analyzed by an HPLC method. Capsules of Example 1 were used as a reference for the dissolution testing.

In vitro dissolution profiles of the reference capsules (Example 1) and capsules from the current invention (Example 2) were compared. The results show that dissolution of the fenofibrate of the current invention is substantially faster than the reference capsules. The difference is not caused by the particle size bias. The particle size of the current invention was larger than that of the reference (Table 1). Therefore, the faster dissolution is not due to a larger surface area of the particles. The increase in dissolution of the current invention is consistent with the function of the effervescent agent. Upon contacting with acidic medium, bicarbonate reacts with the acid to produce bubbles of carbon dioxide gas. The gas bubbles will increase the dispersion of the particles by suspending the particles in the medium, and therefore increase the dissolution rate. The in vitro test condition is similar to the biological condition in a human. Any product will have to contact the acidic gastric fluid after oral administration. The fast dissolution in vitro represents a faster dissolution in the stomach. As disclosed in U.S. Pat. No. 4,895,726, in vitro dissolution can be correlated to in vivo bioavailability in humans. Therefore, faster dissolution in vitro can lead to higher bioavailability in humans.

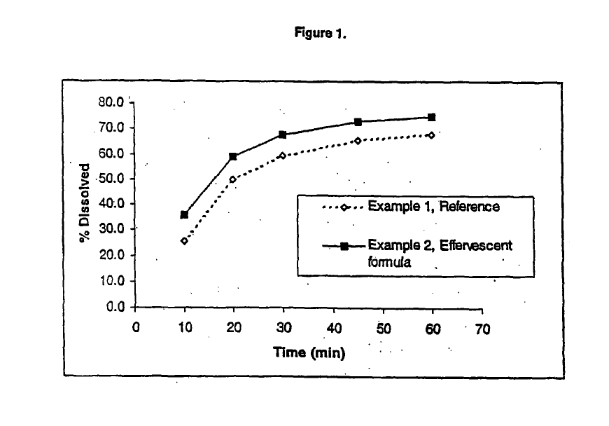

FIG. 1 shows the dissolution profiles of fenofibrate from capsules of a reference composition (Example 1) and those of the current invention (Example 2) (USP II, 50 rpm, 50 mM SDS in 0.1 N HCl, 37° C.). The lower curve represents the slower dissolution of the reference material and the higher curve represents the faster dissolution of the material of the present invention.

Table 1 below shows particle size data. The data is listed for supporting the effect of effervescent agent on the dissolution of fenofibrate. The larger particle size is not required for the current invention. A smaller particle size should increase the dissolution for the current invention.

TABLE 1
Particle size distribution of fenofibrate granules

| | D10 (μg) | D50 (μg) | D90 (μg) | D99 (μg) |
|---|---|---|---|---|
| Example 1 | 3.62 | 26.14 | 186.95 | 390.24 |
| Example 2 | 5.16 | 50.55 | 384.11 | 505.31 |

All references cited are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made in the composition, operation and arrangement of the method of the present invention described herein without departing from the concept and scope of the invention as defined in the following claims:

1. A pharmaceutical composition comprising an effervescent agent and a fibrate.

2. The composition of claim 1 wherein the fibrate is fenofibrate.

3. The composition of claim 1 further comprising pharmaceutical excipients.

4. The composition of claim 2 wherein the effervescent agent is a carbonate.

5. The composition of claim 4 wherein said carbonate is selected from the group consisting of sodium carbonate, sodium bicarbonate, potassium carbonate, potassium bicarbonate, sodium sesquicarbonate, sodium glycine carbonate, L-lysine carbonate, arginine carbonate and calcium carbonate.

6. The composition of claim 3 wherein the pharmaceutical excipients are selected from the group consisting of disintegrants, fillers, lubricants and antiadherents.

7. A process for making the composition of claim 2 wherein said process is selected from the group consisting of a mixing process, a granulation process, a coating process and a melt-congealing process.

8. The process of claim 7 wherein the melt-congealing process comprises the steps of:
incorporating the effervescent agent and other excipients into molten fenofibrate; and
forming a solid dispersion of the effervescent agent and other excipients in fenofibrate that is suitable for manufacturing a finished dosage form.

9. The process of claim 7 wherein the step of forming a solid dispersion is a rotary atomization process, an extrusion process or a milling process.

10. The process of claim 7 further comprising the step of preparing a finished dosage form.

11. The process of claim 10 wherein the finished dosage form is a tablet.

12. The process of claim 10 wherein the finished dosage form is a capsule.

13. A method for lowering lipids comprising the administration to a patient in need of such treatment of an effective amount of the pharmaceutical composition of claim 2.

14. A method for lowering lipids comprising the administration to a patient in need of such treatment of an effective amount of the pharmaceutical composition prepared by the process of claim 7.
